SEQUENCE LISTING (26 sequences)

SEQ ID NO: 1 - DNA, 1017 bp, Escherichia coli
  CDS (1)..(1017)
atggccgttaacttactgaaaaagaactcactcgcgctggtcgcttctctgctgctggcgggccatgtacaggcaacggaactgctgaacagttcttatgacgtctcccgcgagctgtttgccgccctgaatccgccgtttgagcaacaatgggcaaaagataacggcggcgacaaactgacgataaaacaatctcatgccgggtcatcaaaacaggcgctggcgattttacagggcttaaaagccgacgttgtcacttataaccaggtgaccgacgtacaaatcctgcacgataaaggcaagctgatcccggccgactggcagtcgcgcctgccgaataatagctcgccgttctactccaccatgggcttcctggtgcgtaagggtaacccgaagaatatccacgattggaacgacctggtgcgctccgacgtgaagctgattttcccgaacccgaaaacgtcgggtaacgcgcgttatacctatctggcggcatggggcgcagcggataaagctgacggtggtgacaaaggcaaaaccgaacagtttatgacccagttcctgaaaaacgttgaagtgttcgatactggcggtcgtggcgcgaccaccacttttgccgagcgcggcctgggcgatgtgctgattagcttcgaatcggaagtgaacaacatccgtaaacagtatgaagcgcagggctttgaagtggtgattccgaaaaccaacattctggcggaattcccggtggcgtgggttgataaaaacgtgcaggccaacggtacggaaaaagccgccaaagcctatctgaactggctctatagcccgcaggcgcaaaccatcatcaccgactattactaccgcgtgaataacccggaggtgatggacaaactgaaagacaaattcccgcagaccgagctgttccgcgtggaagacaaatttggctcctggccggaagtgatgaaaacccacttcaccagcggcggcgagttagacaagctgttagcggcggggcgtaactga

SEQ ID NO: 2 - protein, 338 aa, Escherichia coli
MAVNLLKKNSLALVASLLLAGHVQATELLNSSYDVSRELFAALNPPFEQQWAKDNGGDKLTIKQSHAGSSKQALAILQGLKADVVTYNQVTDVQILHDKGKLIPADWQSRLPNNSSPFYSTMGFLVRKGNPKNIHDWNDLVRSDVKLIFPNPKTSGNARYTYLAAWGAADKADGGDKGKTEQFMTQFLKNVEVFDTGGRGATTTFAERGLGDVLISFESEVNNIRKQYEAQGFEVVIPKTNILAEFPVAWVDKNVQANGTEKAAKAYLNWLYSPQAQTIITDYYYRVNNPEVMDKLKDKFPQTELFRVEDKFGSWPEVMKTHFTSGGELDKLLAAGRN

SEQ ID NO: 3 - DNA, 834 bp, Escherichia coli
  CDS (1)..(834)
atgtttgctgtctcctccagacgcgtgctgccgggctttaccttaagcctcggcaccagtctgctgtttgtgtgcctgattttgctgctgccgctctccgcgctggtgatgcaactggcccagatgagctgggcgcagtactgggaggtgatcaccaacccgcaggtggtcgcggcctacaaagtaacgctgctgtcggcgtttgtggcatcgatttttaacggcgttttcggtctgctgatggcgtggatcctaacccgctatcgcttcccaggccgcacgctgcttgatgcgctgatggatttaccctttgcgctgccaacggctgtcgccggtttaacgctggcctcgctcttttccgtaaacggtttttacggtgaatggctggcgaagtttgatatcaaagtcacctatacatggctggggattgcggtggctatggcctttaccagcattccgtttgtggtgcgtaccgtgcagccggtgctggaagagttaggcccggaatatgaagaagcggcggaaacgcttggtgcaacgcgctggcagagtttctgcaaagtggtgctgccggagctttctccggcgctggtggcgggcgtggcgctgtcgtttacccgtagtcttggtgaatttggcgcggtgatttttatcgccggaaatatcgcgtggaagacggaagtgacgtcgctgatgatttttgtgcgcttacaggagtttgattacccggcagcgagcgcgattgcttcggtgatcctcgcggcatctctgctgctgctgttctcaattaacactctgcaaagtcgctttggtcggcgtgtggtaggtcattaa

SEQ ID NO: 4 - protein, 277 aa, Escherichia coli
MFAVSSRRVLPGFTLSLGTSLLFVCLILLLPLSALVMQLAQMSWAQYWEVITNPQVVAAYKVTLLSAFVASIFNGVFGLLMAWILTRYRFPGRTLLDALMDLPFALPTAVAGLTLASLFSVNGFYGEWLAKFDIKVTYTWLGIAVAMAFTSIPFVVRTVQPVLEELGPEYEEAAETLGATRWQSFCKVVLPELSPALVAGVALSFTRSLGEFGAVIFIAGNIAWKTEVTSLMIFVRLQEFDYPAASAIASVILAASLLLLFSINTLQSRFGRRVVGH

SEQ ID NO: 5 - DNA, 876 bp, Escherichia coli
  CDS (1)..(876)
atggcggaagttacccaattgaagcgttatgacgcgcgcccgattaactggggcaaatggtttctgattggcatcgggatgctggtttcggcgttcatcctgctggtgccgatgatttacatcttcgtgcaggcattcagcaaggggctgatgccggttttacagaatctggccgatccggacatgctgcacgccatctggctgacggtgatgatcgcgctgattgccgtaccggtaaacctggtgttcggcattctgctggcctggctggtgacgcgctttaacttccctggacgccagttactgctgacgctactggacattccgtttgccgtatcgccggtggttgccggtctggtgtatttgctgttctacggctctaacggcccgctcggcggttggctcgacgagcataacctgcaaattatgttctcctggccgggaatggtgctggtcaccatcttcgtgacgtgtccgtttgtggtgcgcgaactggtgccggtgatgttaagccagggcagccaggaagacgaagcggcgattttgcttggcgcgtccggctggcagatgttccgtcgcgtcacattaccgaacatccgctgggcgctgctttatggcgtggtgttgaccaacgcccgcgcaattggcgagtttggcgcggtgtcggtggtttccggctcgattcgcggcgaaaccctgtcgctgccgttacagattgaattgctggagcaggactacaacaccgtcggctcctttaccgctgcggcgctgttaacgctgatggcgattatcaccctgtttttaaaaagtatgttgcagtggcgcctggagaatcaggaaaaacgcgcacagcaggaggaacatcatgagcattga

SEQ ID NO: 6 - protein, 291 aa, Escherichia coli
MAEVTQLKRYDARPINWGKWFLIGIGMLVSAFILLVPMIYIFVQAFSKGLMPVLQNLADPDMLHAIWLTVMIALIAVPVNLVFGILLAWLVTRFNFPGRQLLLTLLDIPFAVSPVVAGLVYLLFYGSNGPLGGWLDEHNLQIMFSWPGMVLVTIFVTCPFVVRELVPVMLSQGSQEDEAAILLGASGWQMFRRVTLPNIRWALLYGVVLTNARAIGEFGAVSVVSGSIRGETLSLPLQIELLEQDYNTVGSFTAAALLTLMAIITLFLKSMLQWRLENQEKRAQQEEHHEH

SEQ ID NO: 7 - DNA, 1098 bp, Escherichia coli
  CDS (1)..(1098)
atgagcattgagattgccaatattaagaagtcgtttggtcgcacccaggtgctgaacgatatctcactggatattccttcaggtcagatggtcgcgttgctggggccgtccggttccgggaaaaccacgctgctgcgcattatcgccgggctggagcatcaaaccagcgggcatattcgcttccacggcaccgacgtgagccgcctgcacgcacgtgatcgtaaagtcggtttcgtgttccagcattacgcgctgttccgccatatgacggtgttcgacaatatcgcttttggcctgacggtgctgccgcgtcgcgagcgcccgaatgccgcagccatcaaagcgaaagtgacaaaattgctggaaatggtccagcttgcccatctggcggatcgttatccggcgcagctttccggcggccagaaacagcgcgtggcgctggcgcgcgcgctggctgtggaaccgcaaattctgctgcttgatgaaccgtttggcgcgctggatgcgcaggtgcgtaaagagctgcgtcgctggctgcgtcaactccatgaagaactaaaattcaccagcgtttttgtgacccacgatcaggaagaagcgaccgaagtagctgatcgtgtagttgtgatgagccagggcaatattgaacaggctgacgcgccggatcaggtatggcgcgaaccggcgacccgttttgtgctcgaatttatgggcgaagtgaaccgcctgcagggaaccattcgcggcgggcagttccatgttggcgcgcatcgctggccgctgggctacacacctgcgtatcaggggccggtggatctcttcctgcgcccttgggaagtggatatcagccgccgtaccagcctcgattcgccgctgccggtacaggtactggaagccagcccgaaaggtcactacacccaattagtggtgcagccgctggggtggtacaacgaaccgctgacggtcgtgatgcatggcgacgatgccccgcagcgtggcgagcgtttattcgttggtctgcaacatgcgcggctgtataacggcgacgagcgtatcgaaacccgcgatgaggaacttgctctcgcacaaagcgcctga

SEQ ID NO: 8 - protein, 365 aa, Escherichia coli
MSIEIANIKKSFGRTQVLNDISLDIPSGQMVALLGPSGSGKTTLLRIIAGLEHQTSGHIRFHGTDVSRLHARDRKVGFVFQHYALFRHMTVFDNIAFGLTVLPRRERPNAAAIKAKVTKLLEMVQLAHLADRYPAQLSGGQKQRVALARALAVEPQILLLDEPFGALDAQVRKELRRWLRQLHEELKFTSVFVTHDQEEATEVADRVVVMSQGNIEQADAPDQVWREPATRFVLEFMGEVNRLQGTIRGGQFHVGAHRWPLGYTPAYQGPVDLFLRPWEVDISRRTSLDSPLPVQVLEASPKGHYTQLVVQPLGWYNEPLTVVMHGDDAPQRGERLFVGLQHARLYNGDERIETRDEELALAQSA

SEQ ID NO: 9 - DNA, 912 bp, Escherichia coli
  CDS (1)..(912)
gtgagtacattagaacaaacaataggcaatacgcctctggtgaagttgcagcgaatggggccggataacggcagtgaagtgtggttaaaactggaaggcaataacccggcaggttcggtgaaagatcgtgcggcactttcgatgatcgtcgaggcggaaaagcgcggggaaattaaaccgggtgatgtcttaatcgaagccaccagtggtaacaccggcattgcgctggcaatgattgccgcgctgaaaggctatcgcatgaaattgctgatgcccgacaacatgagccaggaacgccgtgcggcgatgcgtgcttatggtgcggaactgattcttgtcaccaaagagcagggcatggaaggtgcgcgcgatctggcgctggagatggcgaatcgtggcgaaggaaagctgctcgatcagttcaataatcccgataacccttatgcgcattacaccaccactgggccggaaatctggcagcaaaccggcgggcgcatcactcattttgtctccagcatggggacgaccggcactatcaccggcgtctcacgctttatgcgcgaacaatccaaaccggtgaccattgtcggcctgcaaccggaagagggcagcagcattcccggcattcgccgctggcctacggaatatctgccggggattttcaacgcttctctggtggatgaggtgctggatattcatcagcgcgatgcggaaaacaccatgcgcgaactggcggtgcgggaaggaatattctgtggcgtcagctccggcggcgcggttgccggagcactgcgggtggcaaaagctaaccctgacgcggtggtggtggcgatcatctgcgatcgtggcgatcgctacctttctaccggggtgtttggggaagagcattttagccagggggcggggatttaa

SEQ ID NO: 10 - protein, 303 aa, Escherichia coli
VSTLEQTIGNTPLVKLQRMGPDNGSEVWLKLEGNNPAGSVKDRAALSMIVEAEKRGEIKPGDVLIEATSGNTGIALAMIAALKGYRMKLLMPDNMSQERRAAMRAYGAELILVTKEQGMEGARDLALEMANRGEGKLLDQFNNPDNPYAHYTTTGPEIWQQTGGRITHFVSSMGTTGTITGVSRFMREQSKPVTIVGLQPEEGSSIPGIRRWPTEYLPGIFNASLVDEVLDIHQRDAENTMRELAVREGIFCGVSSGGAVAGALRVAKANPDAVVVAIICDRGDRYLSTGVFGEEHFSQGAGI

SEQ ID NO: 11 - DNA, 34 bp, Artificial Sequence
  Description of Artificial Sequence primer
agctgagtcgaccgcctcgttatcatccaaaatc

SEQ ID NO: 12 - DNA, 33 bp, Artificial Sequence
  Description of Artificial Sequence primer
agctgagcatgcactaattttccttgcggaggc

SEQ ID NO: 13 - DNA, 32 bp, Artificial Sequence
  Description of Artificial Sequence primer
agctgagcatgctgatggcggcagcacactgc

SEQ ID NO: 14 - DNA, 33 bp, Artificial Sequence
  Description of Artificial Sequence primer
agctgatctagattcactcaacctatcaggcgc

SEQ ID NO: 15 - DNA, 35 bp, Artificial Sequence
  Description of Artificial Sequence primer
agctgagtcgacgtgagtacattagaacaaacaat

SEQ ID NO: 16 - DNA, 33 bp, Artificial Sequence
  Description of Artificial Sequence primer
agctgatctagaagtctccgatgctattaatcc

SEQ ID NO: 17 - DNA, 34 bp, Artificial Sequence
  Description of Artificial Sequence primer
agctgagtcgactccttaactgtatgaaattggg

SEQ ID NO: 18 - DNA, 33 bp, Artificial Sequence
  Description of Artificial Sequence primer
agctgagcatgcccagcctgtttacgatgatcc

SEQ ID NO: 19 - DNA, 33 bp, Artificial Sequence
  Description of Artificial Sequence primer
agctgagtcgacatgtcgcgaaaagatggggtg

SEQ ID NO: 20 - DNA, 33 bp, Artificial Sequence
  Description of Artificial Sequence primer
agctgatctagagtttgttctggccccgacatc

SEQ ID NO: 21 - DNA, 33 bp, Artificial Sequence
  Description of Artificial Sequence primer
agctgagtcgacatgtcgtgtgaagaactggaa

SEQ ID NO: 22 - DNA, 21 bp, Artificial Sequence
  Description of Artificial Sequence primer
atcaccgccgcttcaccaacg

SEQ ID NO: 23 - DNA, 21 bp, Artificial Sequence
  Description of Artificial Sequence primer
cgttggtgaagcggcggtgat

SEQ ID NO: 24 - DNA, 33 bp, Artificial Sequence
  Description of Artificial Sequence primer
agctgatctagaatagatgattacatcgcatcc

SEQ ID NO: 25 - DNA, 33 bp, Artificial Sequence
  Description of Artificial Sequence primer
agctgagtcgacatggcaaaggtatcgctggag

SEQ ID NO: 26 - DNA, 35 bp, Artificial Sequence
  Description of Artificial Sequence primer
agctgatctagattacagcagacgggcgcgaatgg

(19) United States
(12) Patent Application Publication
(10) Pub. No.: US 2005/0124049 A1
(43) Pub. Date: Jun. 9, 2005

(54) Method for producing L-cysteine using bacteria belonging to the genus Escherichia

(76) Inventors: Mikhail Kharisovich Ziyatdinov, Moscow (RU); Ekaterina Igorevna Redkina, Moscow (RU); Mikhail Markovich Gusyatiner, Moscow (RU)

Correspondence Address: CERMAK & KENEALY LLP;ACS LLC, 515 EAST BRADDOCK ROAD, SUITE B, WASHINGTON, DC, 22314, US

(21) Appl. No.: 10/970,986

(22) Filed: Oct. 25, 2004

(30) Foreign Application Priority Data
Nov. 3, 2003 (RU) 2003131993

(51) Int. Cl. C12P 13/12; C12N 1/21

(52) U.S. Cl. 435/113; 435/252.33

(57) ABSTRACT

A method is provided for producing L-cysteine using a bacterium belonging to the genus Escherichia, wherein the L-amino acid productivity of said bacterium is enhanced by increasing expression of the cysPTWAM cluster genes.

### BACKGROUND OF THE INVENTION

1. Field of the Invention

The present invention relates to biotechnology, and specifically to a method for producing L-cysteine by fermentation. The present invention specifically relates to genes derived from Escherichia coli. These genes have been found to be useful for improving L-cysteine production of the E. coli.

2. Description of the Related Art

Conventionally, L-amino acids have been industrially produced by utilizing strains of microorganisms obtained from natural sources or mutants thereof which have been specifically modified to enhance L-amino acid production.

Many techniques have been reported regarding enhancement of L-amino acid production, for example, by transformation of a microorganism by recombinant DNA (see, for example, U.S. Pat. No. 4,278,765). These techniques are based on increasing the activities of the enzymes involved in amino acid biosynthesis and/or desensitizing the target enzymes from the feedback inhibition by the produced L-amino acid (see, for example, U.S. Pat. Nos. 5,661,012 and 6,040,160).

The synthesis of L-cysteine from inorganic sulfur is the predominant mechanism by which reduced sulfur is incorporated into organic compounds in microorganisms, including Salmonella and Escherichia coli. In this process, inorganic sulfate, the most abundant source of utilizable sulfur in the aerobic biosphere, is taken up and reduced to sulfide, which is then incorporated into L-cysteine in a step that is equivalent to the fixation of ammonia into glutamine or glutamate. Sulfate uptake is performed by sulphate permease, which is encoded by the cysTWA and sbp (sulphate binding protein) genes. Two additional mechanisms for sulfur fixation have been described for S. typhimurium and E. coli. The first mechanism occurs through the reaction of thiosulfate with O-acetyl-L-serine catalyzed by O-acetylserine(thiol)-lyase-B encoded by the cysM gene to form the thiosulfonate S-sulfocysteine, which is then reduced to L-cysteine. In this mechanism thiosulphate is transported into the cell by thiosulphate permease, which is encoded by the cysPTWA genes. As alternative, sulfide could be incorporated into O-acetyl-L-serine through the reaction catalyzed by O-acetylserine(thiol)-lyase-A or B, which are encoded by cysK and cysM genes, respectively, yielding L-cysteine. The second mechanism involves the reaction of O-succinyl-L-homoserine with sulfide to form homocysteine in a reaction catalyzed by cystathionine γ-synthase. (Escherichia coli and Salmonella, Second Edition, Editor in Chief: F. C. Neidhardt, ASM Press, Washington D.C., 1996).

A process for the preparation of L-threonine by fermentation of microorganisms of the Enterobacteriaceae family in which at least one or more of the genes of cysteine biosynthesis chosen from cysG, cysB, cysZ, cysK, cysM, cysA, cysW, cysU, cysP, cysD, cysN, cysC, cysJ, cysI, cysH, cysE and sbp is (are) enhanced, in particular over-expressed, has been disclosed (WO03006666A2).

There have been no reports to date, however, describing an improvement in L-cysteine productivity by a bacterium grown in a thiosulphate-containing medium, and wherein the bacterium has been modified to have enhanced expression of the cysPTWAM cluster.

### SUMMARY OF THE INVENTION

An object of the present invention is to enhance the productivity of L-cysteine-producing bacterial strains. It is a further object of the present invention to provide a method for producing the L-cysteine using such strains.

It is a further object of the present invention to provide an L-cysteine producing bacterium belonging to the genus Escherichia, wherein the bacterium has been modified to enhance the expression of the cysPTWAM cluster genes.

It is a further object of the present invention to provide the bacterium as described above, wherein the expression of the cysPTWAM cluster genes is enhanced by increasing the copy number of the cysPTWAM cluster genes or modifying an expression control sequence so that the expression of the genes is enhanced;

It is a further object of the present invention to provide the bacterium as described above, wherein the copy number is increased by transforming the bacterium with a multi-copy vector harboring the cysPTWAM cluster genes.

It is a further object of the present invention to provide the bacterium as described above, wherein the native promoter of said cluster is replaced with more potent promoter.

It is a further object of the present invention to provide the bacterium as described above, wherein the copy number is increased by integrating additional copies of the cysPTWAM cluster genes into chromosome of the bacterium.

It is a further object of the present invention to provide the bacterium as described above, wherein the cysPTWAM cluster genes are derived from a bacterium belonging to the genus Escherichia.

It is a further object of the present invention to provide the bacterium as described above, wherein the bacterium is further modified to have enhanced expression of the ydeD open reading frame.

It is a still further object of the present invention to provide a method for producing L-cysteine, which comprises cultivating the bacterium as described above in a culture medium containing thiosulphate and collecting L-cysteine from the culture medium.

It is a further object of the present invention to provide the method as described above, wherein the bacterium has enhanced expression of L-cysteine biosynthesis genes.

### BRIEF EXPLANATION OF THE DRAWINGS

FIG. 1 shows the relative position of primers cysEF, cysEX-1, cysEX-2 and cysER.

FIG. 2 shows the structure of plasmid pACYC-DES.

FIG. 3 shows the structure of plasmid pMW119int.

### DETAILED DESCRIPTION OF THE INVENTION

The aforementioned objects were achieved by the discovery that enhancing the expression of the cysPTWA cluster genes, as well as the cysM gene, which encode a system for sulphate/thiosulphate transport and O-acetylserine(thiol)-lyase-B, respectively, can enhance L-cysteine production when the novel modified strain described herein is cultivated in a medium containing thiosulphate.

The bacterium of the present invention is an L-cysteine-producing bacterium belonging to the genus Escherichia, which has enhanced expression of the genes of cysPTWAM cluster, which enhances or increases the production yield of L-cysteine. More specifically, the bacterium of the present invention is an L-cysteine-producing bacterium belonging to the genus Escherichia, wherein the bacterium has been modified to enhance expression of the cysPTWAM cluster genes.

“L-cysteine-producing bacterium” means a bacterium, which has an ability to produce and secrete L-cysteine into a medium, when the bacterium is cultured in the medium The term “L-cysteine-producing bacterium” as used herein may also mean a bacterium, which is able to produce and secrete L-cysteine into a culture medium in an amount larger than a wild-type or parental strain, and preferably means that the microorganism is able to produce and secrete L-cysteine into a medium in an amount of at least 0.5 g/L, more preferably at least 1.0 g/L.

The phrase “a bacterium belonging to the genus Escherichia” means that the bacterium is classified as the genus Escherichia according to the classification known to a person skilled in the art of microbiology. A microorganism belonging to the genus Escherichia as used in the present invention includes, but is not limited to Escherichia coli (E. coli). E. coli is one of the most preferred bacterium of the present invention.

The phrase “modified to enhance expression of gene(s)” means that the expression amount of the gene(s) is greater than that of a non-modified strain, for example, a wild-type strain. For example, increasing the number of gene(s) per cell, increasing the number of molecules encoded by the gene(s) per cell, or increasing the gene expression level, and so forth, are encompassed. The copy number of the expressed gene is measured, for example, by restriction of chromosomal DNA followed by Southern blotting using a probe constructed based on the gene sequence, fluorescence in situ hybridization (FISH) and the like. The level of gene expression may be measured by various methods known in the art, including Northern blotting, quantitative RT-PCR, and the like. Furthermore, a wild-type strain, such as Escherichia coli K-12, may serve as a control. As a result of the enhancement of expression of the genes(s), L-cysteine secretion and accumulation in the medium is increased.

Enhancing the expression of the cysPTWAM cluster genes in a bacterial cell can be achieved by increasing the copy number of the cysPTWAM cluster genes or modifying the cluster's expression control sequence so that the expression of the genes is enhanced.

The cysPTWAM cluster genes used in the present invention include those derived from bacteria belonging to the genus Escherichia, as well as those derived from other bacteria, such as Salmonella. Genes derived from bacteria belonging to the genus Escherichia are preferred.

Sequences of the cysPTWAM cluster genes from Escherichia coli have been reported as follows (Blattner, F. R. et al, Science, 277 (5331), 1453-1474 (1997)):
cysP gene (SEQ ID NO: 1)-nucleotide numbers 2540532 to 2541548 in the sequence of GenBank accession NC_{—}000913.1 (gi: 16130350),
cysT (cysU) gene (SEQ ID NO: 3)-nucleotide numbers 2539699 to 2540532 in the sequence of GenBank accession NC_{—}000913.1 (gi: 16130349),
cysW gene (SEQ ID NO: 5)-nucleotide numbers 2538824 to 2539699 in the sequence of GenBank accession NC_{—}000913.1 (gi: 16132224),
cysA gene (SEQ ID NO: 7)-nucleotide numbers 2537737 to 2538834 in the sequence of GenBank accession NC_{—}000913.1 for (gi: 16130348),
cysM gene (SEQ ID NO: 9)-nucleotide numbers 2536692 to 2537603 in the sequence of GenBank accession NC_{—}000913.1 (gi: 16130347).

The cysPTWAM cluster is located between ychM ORF b2420 locus and b2426 locus on the chromosome of E. coli strain K-12. Therefore, these genes can be obtained by PCR (polymerase chain reaction; refer to White, T. J. et al., Trends Genet., 5, 185 (1989)) utilizing primers based on the reported nucleotide sequences of the genes. Genes encoding the proteins of sulphate/thiosulphate transport system which are derived from other microorganisms can be obtained in a similar manner.

Examples of the genes of the cysPTWAM cluster derived from Escherichia coli include following DNAs:
the cysP gene which encodes the protein (A) or (B):
   (A) a protein having the amino acid sequence shown in SEQ ID NO: 2; or
   (B) a protein variant of the amino acid sequence shown in SEQ ID NO: 2, which exhibits an activity of thiosulphate periplasmic binding protein;
the cysT gene which encodes the protein (C) or (D):
   (C) a protein having the amino acid sequence shown in SEQ ID NO: 4; or
   (D) a protein variant of the amino acid sequence shown in SEQ ID NO: 4, which exhibits an activity of sulphate/thiosulphate transport system permease when combined with proteins (E) or (F), and (G) or (H);
the cysW gene which encodes the protein (E) or (F):
   (E) a protein having the amino acid sequence shown in SEQ ID NO: 6; or
   (F) a protein variant of the amino acid sequence shown in SEQ ID NO: 6, which exhibits an activity of sulphate/thiosulphate transport system permease when combined with proteins (C) or (D), and (G) or (H);
the cysA gene which encodes the protein (G) or (H):
   (G) a protein having the amino acid sequence shown in SEQ ID NO: 8; or
   (H) a protein variant of the amino acid sequence shown in SEQ ID NO: 8, which is a ATP-binding component of sulphate/thiosulphate permease and exhibits an activity of the sulphate/thiosulphate transport system permease when combined with proteins (C) or (D), and (E) or (F);
the cysM gene which encodes the protein (I) or (J):
   (I) a protein having the amino acid sequence shown in SEQ ID NO: 10; or
   (J) a protein variant of the amino acid sequence shown in SEQ ID NO: 10, which exhibits an activity of O-acetylserine(thiol)-lyase-B.

The phrase “an activity of sulphate/thiosulphate transport system permease” means an activity of a protein which transports thiosulphate into the cell from the outer medium. The phrase “an activity of O-acetylserine(thiol)-lyase-B” means an activity which catalyzes the reaction between O-acetyl-L-serine and thiosulphate, yielding S-sulfocysteine. The presence of these activities may be determined by, for example, complementation experiments using bacteria having mutations in the corresponding genes.

The DNA encoding proteins of the present invention includes a DNA encoding protein variants, possibly having deletions, substitutions, insertions or additions of one or several amino acids in one or more positions in the proteins (A), (C), (E), (G) or (I), as long as such changes do not result in loss of the protein's activity. The number of “several” amino acids differs depending on the position of amino acid residues in the three-dimensional structure of the protein and the type of the amino acids. However, it preferably means between 2 to 30, more preferably between 2 to 20, and most preferably between 2 to 10 for a protein having approximately 300 amino acid residues. This is because some amino acids have high homology to one another and the differences between the amino acid sequences does not greatly affect the three dimensional structure of the protein and its activity. Therefore, the protein (B), (D), (F), (H) or (J) may be one which has homology of not less than 30 to 50%, preferably 50 to 70%, more preferably 70 to 90%, more preferably not less than 90%, and most preferably not less than 95% with respect to the entire amino acid sequence of the protein (A), (C), (E), (G) or (I), respectively, and which has the activity of the respective protein.

To evaluate the degree of protein or DNA homology, known calculation methods can be used, such as BLAST search, FASTA search and CrustalW.

BLAST (Basic Local Alignment Search Tool) is the heuristic search algorithm employed by the programs blastp, blastn, blastx, megablast, tblastn, and tblastx; these programs ascribe significance to their findings using the statistical methods of Karlin, Samuel and Stephen F. Altschul (“Methods for assessing the statistical significance of molecular sequence features by using general scoring schemes”. Proc. Natl. Acad. Sci. USA, 1990, 87:2264-68; “Applications and statistics for multiple high-scoring segments in molecular sequences”. Proc. Natl. Acad. Sci. USA, 1993, 90:5873-7). FASTA search method described by W. R. Pearson (“Rapid and Sensitive Sequence Comparison with FASTP and FASTA”, Methods in Enzymology, 1990 183:63-98). ClustalW method described by Thompson J. D., Higgins D. G. and Gibson T. J. (“CLUSTAL W: improving the sensitivity of progressive multiple sequence alignment through sequence weighting, position-specific gap penalties and weight matrix choice”, Nucleic Acids Res. 1994, 22:4673-4680).

Changes to the protein defined in (A), (C), (E), (G) or (I) such as those described above are typically conservative changes so as to maintain the activity of each protein. Substitution changes include those in which at least one residue in the amino acid sequence has been removed and a different residue inserted in its place. Examples of amino acids which may be substituted for an original amino acid in the above protein and which are regarded as conservative substitutions include: Ala substituted with ser or thr; arg substituted with gin, his, or lys; asn substituted with glu, gin, lys, his, asp; asp substituted with asn, glu, or gin; cys substituted with ser or ala; gin substituted with asn, glu, lys, his, asp, or arg; glu substituted with asn, gin, lys, or asp; gly substituted with pro; his substituted with asn, lys, gin, arg, tyr; ile substituted with leu, met, val, phe; leu substituted with ile, met, val, phe; lys substituted with asn, glu, gin, his, arg; met substituted with ile, leu, val, phe; phe substituted with trp, tyr, met, ile, or leu; ser substituted with thr, ala; thr substituted with ser or ala; trp substituted with phe, tyr; tyr substituted with his, phe, or trp; and val substituted with met, ile, leu.

The DNA encoding substantially the same proteins as the protein defined in (A), (C), (E), (G) or (I), such as a protein variant, may be obtained by, for example, modification of the nucleotide sequence encoding the protein defined in (A), (C), (E), (G) or (I) using site-directed mutagenesis so that one or more amino acid residue will be deleted, substituted, inserted or added. This modified DNA can be obtained by conventional methods of treatment with reagents under conditions which typically generate mutations. These treatments include treating the DNA which encodes proteins of present invention with hydroxylamine, or treating the bacterium harboring the DNA with UV irradiation or a reagent such as N-methyl-N′-nitro-N-nitrosoguanidine or nitrous acid.

The DNA of the cysPTWAM cluster genes include variants derived from different strains and variants of bacteria belonging to the genus Escherichia by virtue of natural diversity.

DNA encoding such variants can be obtained by isolating DNA which hybridizes to the cysP, cysT, cysW, cysA or cysM gene or a part thereof under stringent conditions, and which encodes the protein having an inherent activity of the protein encoded by each of the genes. The term “stringent conditions” may include conditions under which a so-called specific hybrid is formed, and a non-specific hybrid is not formed. For example, stringent conditions include conditions under which DNAs having high homology, for instance DNAs having homology not less than 70%, preferably not less than 80%, more preferably not less than 90%, most preferably not less than 95% to each other, are able to hybridize. Alternatively, stringent conditions may include typical washing conditions for Southern hybridization, e.g., 60° C., 1×SSC, 0.1% SDS, preferably 0.1×SSC, 0.1% SDS. Duration of the washing procedure depends on the type of membrane used for blotting and, as a rule, is recommended by manufacturer. For example, recommended duration of washing the Hybond™ N+ nylon membrane (Amersham) under stringent conditions is 15 minutes. As a probe for the DNA that encodes variants and hybridizes with the cysP, cysT, cysW, cysA or cysM gene, a partial sequence of the nucleotide sequence of SEQ ID NO: 1, 3, 5, 7 or 9 can also be used. Such a probe may be prepared by PCR using oligonucleotides based on the nucleotide sequence of SEQ ID NO: 1, 3, 5, 7 or 9 as primers, and a DNA fragment containing the nucleotide sequence of SEQ ID NO: 1, 3, 5, 7 or 9 as a template. When a DNA fragment of about 300 bp in length is used as the probe, the washing conditions for the hybridization can be, for example, 50° C., 2×SSC, and 0.1% SDS.

Methods for enhancing gene expression include increasing the gene copy number. Introducing a gene into a vector that is able to function in a bacterium belonging to the genus Escherichia will increase the copy number of the gene. Multi-copy vectors can be preferably used, and include pBR322, pUC 19, pBluescript KS⁺, pACYC 177, pACYC 184, pAYC32, pMW119, pET22b and the like. Enhancing gene expression can be achieved by introducing multiple copies of the gene into the bacterial chromosome via, for example, homologous recombination methods and the like.

Transforming a bacterium with a DNA harboring a gene encoding a protein means introduction of the DNA into the bacterium, for example, by conventional methods, to increase expression of the gene in the bacterium.

Furthermore, enhancing gene expression can be achieved by placing the DNA of the present invention under the control of a more potent promoter rather than the native promoter. The term “native promoter” means a DNA region which is present in a wild-type organism, located upstream of the open reading frame (ORF) of the gene, and promotes expression of the gene. Translational coupling of genes in the cysPTWA cluster indicates that these genes are expressed as a single transcription unit from a promoter just upstream of cysP gene (Hryniewicz, M. M., Kredich, N. M., J. Bacteriol., 173(18), 5876-86 (1991)). cysM gene is separated from cysA gene by only 174 bp in the chromosome of E. coli and may also be part of this operon, which is transcribed counterclockwise on the chromosome (Escherichia coli and Salmonella, Second Edition, Editor in Chief: F. C. Neidhardt, ASM Press, Washington D.C., 1996). Promoter strength is defined as the frequency of acts of RNA synthesis initiation. Methods for evaluating the strength of a promoter are described by, for example, Deuschle U., Kammerer W., Gentz R., Bujard H. (Promoters in Escherichia coli: a hierarchy of in vivo strength indicates alternate structures. EMBO J. 1986, 5, 2987-2994).

Enhancing translation can also be achieved by introducing into the DNA of the present invention a more efficient Shine-Dalgarno sequence (SD sequence). The SD sequence is a region upstream of the start codon of mRNA which interacts with the 16S RNA of ribosome (Shine J. and Dalgarno L., Proc. Natl. Acad. Sci. USA, 1974, 71, 4, 1342-6). The term “native SD sequence” means the SD sequence present in the wild-type organism. An example of an efficient SD sequence includes the SD sequence of the φ10 gene from phage T7 (Olins P. O. et al, Gene, 1988, 73, 227-235).

Use of potent promoters can be combined with multiplication of gene copies. It is also possible to increase the copy number of genes of cysPTWAM cluster by combining the integration of one or several genes of the cluster with introduction of one of several genes into a multi-copy vector.

Methods for preparation of chromosomal DNA, hybridization, PCR, preparation of plasmid DNA, digestion and ligation of DNA, transformation, selection of an oligonucleotide as a primer and the like include typical methods well known to one of ordinary skill in the art. Such methods are described in Sambrook, J., and Russell D., “Molecular Cloning A Laboratory Manual, Third Edition”, Cold Spring Harbor Laboratory Press (2001) and the like.

The bacterium of the present invention can be obtained by introduction of the aforementioned DNAs into a bacterium which inherently has the ability to produce L-cysteine. Alternatively, the bacterium of present invention can be obtained by imparting the ability to produce L-cysteine to the bacterium already harboring the DNAs.

The parent strain which is to be modified to have enhanced activity of the protein of the present invention may include an L-cysteine-producing bacterium belonging to the genus Escherichia, such as E. coli strain JM15 which is transformed with different cysE alleles encoding feedback-resistant serine acetyltransferases (U.S. Pat. No. 6,218,168, Russian patent application 2003121601); E. coli strain W3110 having overexpressed genes which encode a protein able to secrete toxic substances from cells (U.S. Pat. No. 5,972,663); E. coli strains with low cysteine desulfhydrase activity (JP11155571A2); E. coli strain W3110 with increased activity of a positive transcriptional regulator for cysteine regulon coded by cysB gene (PCT application WO0127307A1) and the like.

It has been reported that the ydeD gene, which encodes a membrane protein not involved in the biosynthetic pathway of any L-amino acid, can enhance production of L-cysteine when additional copies of the gene are introduced into cells of the respective producing strain (U.S. Pat. No. 5,972,663). Therefore, an embodiment of the present invention includes the L-cysteine-producing bacterium which is further modified to have enhanced expression of the ydeD open reading frame.

The bacterium of the present invention may also have enhanced expression of L-cysteine biosynthesis genes. Such genes include the cysE gene, which encodes feed-back resistant serine acetyltransferase (6,218,168), the serA gene, which encodes feed-back resistant phosphoglycerate dehydrogenase (U.S. Pat. No. 6,180,373), and the like.

Proteins encoded by the ydeD, cysE or serA genes may be variants in the same manner as described for the cysP, cysT, cysW, cysA or cysM gene products. The method of present invention includes production of L-cysteine comprising the steps of cultivating the bacterium of the present invention in a culture medium, allowing the L-cysteine to be produced by the bacterium and secreted, and collecting the L-cysteine from the culture medium.

In the present invention, the cultivation, collection and purification of L-cysteine from the medium and the like may be performed by conventional fermentation and purification methods typically used for production and isolation of an amino acid using a microorganism.

The medium used for culture may be either a synthetic medium or a natural medium, so long as the medium includes a carbon source, a nitrogen source, a sulphur source and minerals and, if necessary, appropriate amounts of nutrients which the chosen microorganism requires for growth.

The carbon source may include various carbohydrates such as glucose and sucrose, and various organic acids. Depending on the mode of assimilation of the chosen microorganism, alcohol, including ethanol and glycerol, may be used.

As the nitrogen source, various ammonium salts such as ammonia and ammonium salts, other nitrogen compounds such as amines, a natural nitrogen source such as peptone, soybean-hydrolysate and digested fermentative microorganism can be used.

Thiosulphates may be used as the sulphur source for the present invention.

Potassium monophosphate, sodium chloride, calcium chloride, magnesium salts, ferrous salts, manganese salts and the like may be used as minerals.

Some additional nutrients can be added to the medium if necessary. For instance, if the microorganism requires methionine for growth (methionine auxotrophy), a sufficient amount of methionine can be added to the medium for cultivation.

The cultivation is preferably performed under aerobic conditions such as by shaking, and/or stirring with aeration, at a temperature of 20 to 42° C., preferably 34 to 40° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to the production, secretion, and accumulation of L-cysteine in the liquid medium.

After cultivation, solids such as cells can be removed from the liquid medium by centrifugation or membrane filtration, and the L-cysteine can be collected and purified by methods such as ion-exchange, concentration and/or crystallization.

### EXAMPLES

The present invention will be more concretely explained with reference to the following non-limiting Examples. In the Examples an amino acid is of L-configuration unless otherwise noted. Chromosomal DNA of E. coli strain MG1655 (VKPM B-6195) was used as a template in all PCRs.

### Example 1

### Construction of the Plasmid Carrying ydeD Gene, Mutant cysE Gene and Mutant serA5 Gene

It has been reported that the ydeD gene, which encodes a transmembrane protein, is useful for cysteine production (U.S. Pat. No. 5,972,663). Therefore, the ydeD gene was cloned by PCR using primers ydeD299F and ydeD299R (SEQ ID NOS: 19 and 20, respectively) and chromosomal DNA from E. coli strain MG1655.

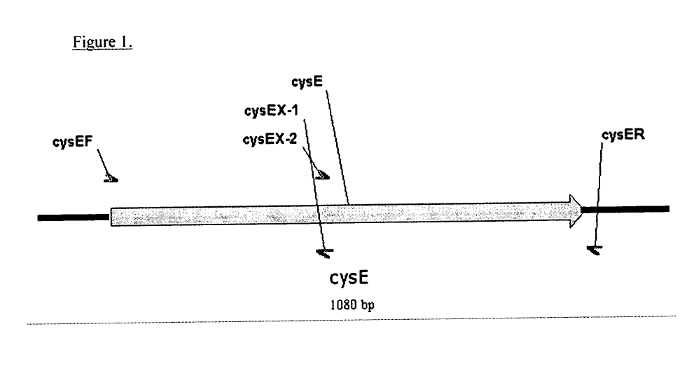

Then, mutant cysEX gene, which encodes serine acetyltransferase free from feedback inhibition by cysteine described in the U.S. Pat. No. 6,218,168, was constructed by two successive PCR procedures for site-directed mutagenesis using primers cysEF and cysEX-1, cysEX-2 and cysER (SEQ ID NOS: 21 and 22, 23 and 24, respectively) and chromosomal DNA from E. coli strain MG1655 as it shown on FIG. 1. The two resulting PCR products were separated by electrophoresis and eluted from gel. In the second PCR these two DNA fragments were annealed and mutant cysEX gene was completed.

Also, the mutant serA5 gene, which encodes the phosphoglycerate dehydrogenase free from feedback inhibition by serine described in the U.S. Pat. No. 6,180,373, was cloned by PCR using primers serA5F and serA5R (SEQ ID NOS: 25 and 26, respectively) and chromosomal DNA from E. coli strain MG1655. Serine is the precursor of L-cysteine, so amplification of the mutant serA5 gene is necessary to increase the amount of serine.

Finally, promoter P_{ompA} was cloned by PCR using primers depicted in SEQ ID No: 11 (primer PrOMPAF) and No. 12 (primer PrOMPAR). The primer PrOMPAF contains a restriction enzyme SalI recognition site which has been introduced at the 5′-end thereof. A SalI site was also introduced into forward primers for amplification of ydeD (SEQ ID NO: 19), cysEX (SEQ ID NO: 21) and serA5 (SEQ ID NO: 25) genes. So, the SalI site was used for assembling promoter P_{ompA} and each of genes. The primer PrOMPAR contains a restriction enzyme PaeI recognition site introduced at the 5′-end thereof. These restriction sites were introduced for further assembly of the genes and construction of a plasmid which is used for transformation.

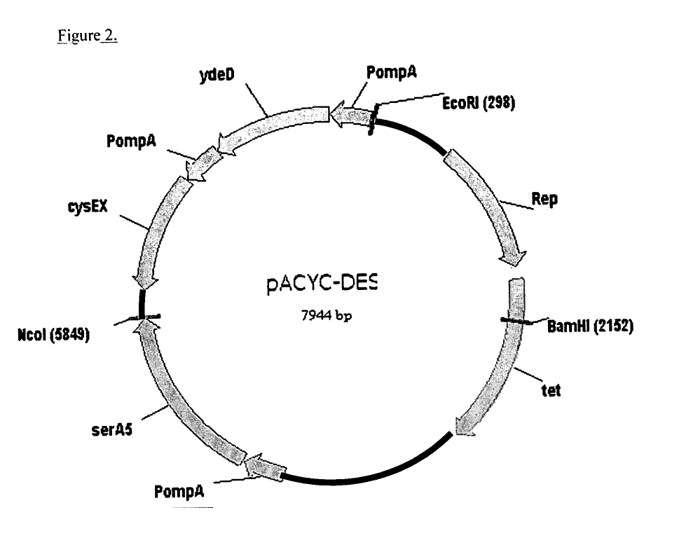

Then all three genes, each under promoter P_{ompA}, were cloned into vector pACYC184. Thus, the plasmid pACYC-DES was obtained (FIG. 2).

### Example 2

### Cloning of the cysPTWA Genes from E. coli

The cysPTWA genes, which encode proteins of the sulphate/thiosulphate transport system, were cloned using the primers depicted in SEQ ID No: 13 (primer Mz025) and No. 14 (primer Mz026). The primer Mz025 is identical to a sequence starting 315 bp upstream of the start codon of the cysP gene, and having a restriction enzyme PaeI recognition site introduced at the 5′-end thereof. The primer Mz026 is a sequence complementary to a sequence starting 13 bp downstream of the termination codon of cysA gene and having a restriction enzyme SalI recognition site introduced at the 5′-end thereof. The resulting PCR fragment, which contains the cysPTWA cluster under its own promoter, was treated with PaeI and SalI restrictases and inserted into vector pMW119, which had been previously treated with the same enzymes. Thus plasmid pMW-PTWA was obtained.

### Example 3

### Cloning of the cysM Gene from E. coli

The cysM gene encoding O-acetylserine(thiol)-lyase-B was cloned by PCR using the primers depicted in SEQ ID No: 15 (primer cysMF) and No. 16 (primer cysMR). The primer cysMF contains a restriction enzyme SalI recognition site which has been introduced at the 5′-end thereof. The primer cysMR contains a restriction enzyme XbaI recognition site which has been introduced at the 5′-end thereof.

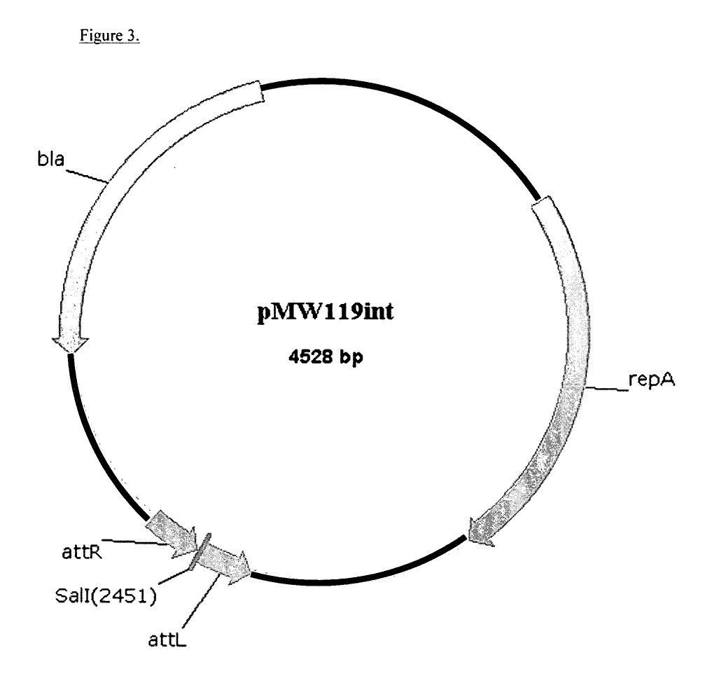

Promoter P_{cysK} was obtained by PCR using primers depicted in SEQ ID No: 17 (primer PcyskF) and No. 18 (primer PcysKR). The primer PcysKF is identical to a sequence starting 6 bp upstreamz of the start codon of cysK gene, and a restriction enzyme SalI recognition site has been introduced at the 5′-end thereof. The primer PcysKR is complementary to a sequence starting 301 bp upstream of the start codon of cysK gene, and a restriction enzyme PaeI recognition site has been introduced at the 5′-end thereof. Then, the two resulting PCR products were assembled by treatment with restrictase SalI followed by ligation, and introduced into the integrative vector pMW119int. Vector pMW119int (FIG. 3) was constructed from the commercially available vector pMW119 by insertion of attR and attL sites, which are necessary for further Mu-integration into SalI site of pMW119. Thus plasmid pMW-P_{cysK}-cysM was obtained.

### Example 4

### Construction of the E. coli Strain with an Altered System for L-Cysteine Degradation

The tnaA and metC genes encode proteins of the main cysteine degradation pathway (Japanese patent application JP2002271463). Therefore, an E. coli strain MG1655 with an altered L-cysteine degradation (tnaA⁻, metc⁻) system was constructed as follows.

Initially, a mutation in the metC gene was induced by N-methyl-N′-nitro-N-nitrosoguanidine (NTG) treatment followed by multiple procedures of ampicillin enrichment. The mutant, which was able to grow on cystathionine, but not on homocysteine, was selected. Then, the disrupted tnaA gene from the strain VKPM B-7427 (tnaA300::Tn10(Tc^{R})) was transferred into the resulting metC⁻ strain by the standard procedure of P1 transduction (Sambrook et al, “Molecular Cloning A Laboratory Manual, Second Edition”, Cold Spring Harbor Laboratory Press (1989)).

Thus the E. coli strain MT was obtained.

### Example 5

### Effect of Enhanced Expression of cysPTWAM Cluster on L-Cysteine Production

The E. coli strain MT was used as a parental strain to evaluate the effect of enhanced expression of the cysPTWAM cluster on L-cysteine production.

Initially, the cysM gene was integrated into the chromosome of strain MT using the plasmid pMW-P_{cysK}-cysM by the standard procedure of Mu-integration.

Then, plasmids pACYC-DES and pMW-PTWA were subsequently introduced into the resulting transductant MTintCYSM, giving strains MTintCYSM/pACYC-DES and MTintCYSM/pACYC-DES/pMW-PTWA.

Both strains MTintCYSM/pACYC-DES and MTintCYSM/pACYC-DES/pMW-PTWA were cultivated overnight with shaking at 34° C. in 2 ml of nutrient broth which had been supplemented with 50 mg/l of ampicillin and 20 μg/ml of tetracycline. 0.2 ml of the resulting cultures were inoculated into 2 ml of a fermentation medium containing tetracycline (20 mg/l) and ampicillin (50 mg/l) in 20×200 mm test tubes, and cultivated at 34° C. for 42 hours with a rotary shaker at 250 rpm. The composition of the fermentation medium was 15.0 g/l of (NH₄)₂SO₄, 1.5 g/l of KH₂PO₄, 1.0 g/l of MgSO₄, 20.0 g/l of CaCO₃, 0.1 mg/l of thiamine, 1% of Luria broth (LB medium), 4% of glucose, 300 mg/l of L-methionine and varied concentrations of Na₂S₂O₃.

After the cultivation, the amount of L-cysteine which had accumulated in the medium was determined by the method described by Gaitonde, M. K. (Biochem. J., 104:2, 627-33 (1967)). Data are presented in Table 1.

Table 1

| | Thiosulphate | Amount of |
|---|---|---|
| Strains | concentration, g/l | L-cysteine, g/l |
| MTintCYSM/pACYC-DES | 2.5 | 3.1 |
| | 5.0 | 3.4 |
| | 7.0 | 3.8 |
| | 10.0 | 2.8 |
| MTintCYSM/pACYC-DES/ | 2.5 | 4.1 |
| pMW-PTWA | 5.0 | 5.6 |
| | 7.0 | 6.2 |
| | 10.0 | 5.5 |

For mini-jar fermentation, one loop of each strain MTintCYSM/pACYC-DES and MTintCYSM/pACYC-DES/pMW-PTWA grown on L-agar was transferred to L-broth and cultivated at 34° C. with rotation (140 rpm) to reach optical density of culture OD₅₄₀≈2.0. Then 25 ml of seed culture was added to 250 ml of medium for fermentation and cultivated at 34° C. with rotation (1500 rpm) for 48 hours.

The composition of the fermentation medium for jar-fermenter (g/l):

| | |
|---|---|
| Tryptone | 2.0 |
| Yeast extract | 1.0 |
| (NH₄)₂SO₄ | 5.0 |
| KH₂PO₄ | 1.5 |
| NaCl | 0.5 |
| MgSO₄.7H₂O | 0.3 |
| CaCl₂.2H₂O | 0.015 |
| FeSO₄.7H₂O | 0.075 |
| Sodium citrate.2H₂O | 1.0 |
| Glucose | 100.0 |
| Methionine | 0.45 |

The medium was supplemented with thiosulfate after 6 hours of fermentation at a velocity of 0.4 g/l*h.

After the cultivation, the amount of L-cysteine which had accumulated in the medium was determined as described above. Data are presented in the Table 2.

TABLE 2

| | Amount of |
|---|---|
| Strains | L-cysteine, g/l |
| MTintCYSM/pACYC-DES | 3.9 |
| MTintCYSM/pACYC-DES/pMW-PTWA | 6.6 |

As seen in Tables 1 and 2, enhanced expression of genes from cysPTWAM cluster improved cysteine productivity of the MT strain.

While the invention has been described in detail with reference to preferred embodiments thereof, it will be apparent to one skilled in the art that various changes can be made, and equivalents employed, without departing from the scope of the invention. Each of the aforementioned documents, as well as the foreign priority document, RU2003131993, is incorporated by reference herein in its entirety.

1. An L-cysteine-producing bacterium belonging to the genus Escherichia, wherein the bacterium has been modified to enhance the expression of cysPTWAM cluster genes.

2. The bacterium according to claim 1, wherein the expression of said cysPTWAM cluster genes is enhanced by increasing the copy number of the cysPTWAM cluster genes or modifying an expression control sequence so that the expression of said genes isenhanced;

3. The bacterium according to claim 2, wherein the copy number is increased by transforming the bacterium with a multi-copy vector harboring cysPTWAM cluster genes.

4. The bacterium according to claim 2, wherein the native promoter of said cluster is replaced with a more potent promoter.

5. The bacterium according to claim 2, wherein the copy number is increased by integrating additional copies of said cysPTWAM cluster genes into the chromosome of the bacterium.

6. The bacterium according to claim 1, wherein said cysPTWAM cluster genes are derived from a bacterium belonging to the genus Escherichia.

7. The bacterium according claim 6, wherein the bacterium is further modified to have enhanced expression of the ydeD open reading frame.

8. A method for producing L-cysteine which comprises cultivating the bacterium according to claim 1 in a culture medium containing thiosulphate, and collecting L-cysteine from the culture medium.

9. The method according to claim 8, wherein the bacterium has enhanced expression of L-cysteine biosynthesis genes.
